(19) United States
(12) Patent Application Publication
(10) Pub. No.: US 2011/0144541 A1
(43) Pub. Date: Jun. 16, 2011

(54) BIOMETRIC DATA-MEASURING INSTRUMENT, BIOMETRIC DATA-MEASURING SYSTEM, MUSCLE STRENGTH METER, AND MUSCLE STRENGTH-MEASURING SYSTEM

(75) Inventors: Hisashi KURODA, Tokyo (JP); Makoto Sasaki, Tokyo (JP)

(73) Assignee: ITO CO., LTD., TOKYO (JP)

(21) Appl. No.: 12/964,637

(22) Filed: Dec. 9, 2010

(30) Foreign Application Priority Data
Dec. 10, 2009 (JP) 2009-280666

(51) Int. Cl. A61B 5/103

(52) U.S. Cl. 600/587

(57) ABSTRACT

A biometric data-measuring instrument that measures data relating to a living organism by applying pressure to the living organism. The instrument includes: a casing, an auxiliary contacting part that extends from the casing, the auxiliary contacting part being contacted against a vicinity of a point to be measured on the living organism and applying pressure to the vicinity of the point to be measured, a main contacting part that, in a state where the auxiliary contacting part is applying pressure to the vicinity of the point to be measured, is contacted against the point to be measured and applies pressure to the point to be measured in the direction in which the auxiliary contacting part is applying pressure to the vicinity of the point to be measured, a pressure sensor that is provided inside the casing and measures a pressure that the main contacting part receives from the point to be measured, and a biometric data display unit that is provided on the casing and displays the measured biometric data. A tip of the auxiliary contacting part extends outward from a base side thereof.

### BACKGROUND OF THE INVENTION

1. Field of the Invention

The present invention relates to a biometric data-measuring instrument and a biometric data-measuring system that measure biometric data such as the tenderness and hardness of a muscle tissue of a living organism, a muscle strength meter, and a muscle strength-measuring system.

Priority is claimed on Japanese Patent Application No. 2009-280666, filed on Dec. 10, 2009, the contents of which are incorporated herein by reference.

2. Background Art

A muscle hardness meter including a detector having a probe which contacts a living organism, and a device main body that performs various computations and displays muscle hardness is known in the related art (e.g. refer to Japanese Patent Application, First Publication No. 2008-272286.

In the related art there is a muscle strength meter including an attachment that includes a contacting part that contacts to the living organism and an attaching shaft part provided on the contacting part, a detector that includes an insertion hole for inserting the attaching shaft part therein and a pressure sensor built into a tip of the insertion hole, and a device main body that performs various types of computations to an input signal from the detector. Using this type of muscle strength meter, in a state where the attaching shaft part is inserted in the insertion hole, the contacting part is made to contact the living organism; when the contacting part is pressed using muscle strength, the sensor is also pressed via the attaching shaft part, and the device main body performs various types of computations to this detection result to display the muscle strength. A muscle strength meter of a different type to this one is disclosed in, for example, Japanese Patent Application, First Publication No. 2004-180982.

However, the biometric data-measuring instrument and the muscle strength meter described above include, in addition to a detector having a probe and an attachment that contacts to the living organism, a device main body for displaying the measurement result. The whole devices of the biometric data-measuring instrument and the muscle strength meter described above are consequently bulky, making them inconvenient for carrying.

The present invention has been realized after consideration of these problems, and aims to provide a biometric data-measuring instrument and a biometric data-measuring system, a muscle strength meter, and a muscle strength-measuring system with excellent portability.

### DISCLOSURE OF THE INVENTION

To achieve the above problems and achieve the objects, the present invention employs the following means.

A biometric data-measuring instrument according to the invention measures data relating to a living organism by applying pressure to the living organism. The biometric data-measuring instrument includes: a casing, an auxiliary contacting part that extends from the casing, the auxiliary contacting part being contacted against a vicinity of a point to be measured on the living organism and applying pressure to the vicinity of the point to be measured, a main contacting part that, in a state where the auxiliary contacting part is applying pressure to the vicinity of the point to be measured, is contacted against the point to be measured and applies pressure to the point to be measured in the direction in which the auxiliary contacting part is applying pressure to the vicinity of the point to be measured, a pressure sensor that is provided inside the casing and measures a pressure that the main contacting part receives from the point to be measured, a biometric data display unit that is provided on the casing and displays the measured biometric data. A tip of the auxiliary contacting part extends outward from a base side thereof.

According to this configuration, since the casing includes the biometric data display unit for displaying measured biometric data (e.g. tissue hardness), there is no need to provide a separate device including the biometric data display unit. The configuration of the whole biometric data-measuring instrument can thus be made compact. Therefore, the portability of the biometric data-measuring instrument can be enhanced.

Furthermore, since the tip of the auxiliary contacting cylinder part extends further outward than the base side, the auxiliary contacting part is easily contacted against a point to be measured. Since this makes it possible to considerably adjust the relative angle of the living organism and the biometric data-measuring instrument even if the point to be measured cannot easily be visually confirmed with biometric data display unit, a reduction in visibility from the biometric data display unit can be suppressed.

The biometric data display unit can be facing in the pressure-receiving direction in which the auxiliary contacting part receives pressure from the living organism.

According to this configuration, since the biometric data display unit is facing in the pressure-receiving direction, when the person who is measuring is positioned in the pressure-receiving direction with respect to the point to be measured, he can easily visually confirm the biometric data.

The casing can have a grasp part extending in a direction intersecting the pressure-applying direction, with the biometric data display unit facing in an opposite direction to a direction in which the grasp part extends.

According to this configuration, since the casing has a grasp part extending in a direction intersecting the pressure-applying direction, the orientation of the casing can be stabilized by grasping the grasp part such that it is in a vertical plane. This makes it possible to apply pressure stably to the point to be measured, and to more accurately measure the biometric data.

Furthermore, since the biometric data display unit is facing in the opposite direction to the direction in which the grasp part extends, when the biometric data display unit is below the eye level of the person who is measuring, he can easily visually confirm the muscle hardness and tenderness.

The auxiliary contacting part can include a detachable extending part that is detachably provided at a tip of the auxiliary contacting part, and a locking mechanism at a tip of this detachable extending part.

According to this configuration, since the auxiliary contacting part includes the detachable extending part that is detachably provided at the tip, and the locking mechanism, the portability of the biometric data-measuring instrument can be further enhanced.

Furthermore, the biometric data-measuring instrument can include a securing mechanism that secures the auxiliary contacting part such that the tip of the main contacting part is positioned further to the pressure-applying direction side than the tip of the auxiliary contacting part, and a switch that, when switched on, makes the biometric data display unit display the measured biometric data.

According to this configuration, since the instrument includes the securing mechanism that secures the auxiliary contacting part in the pressure-applying direction, and the switch, it can measure, for example, biometric data such as tenderness (pain threshold). This enables the biometric data-measuring instrument to function as a pressure algometer.

A biometric data-measuring system according to the present invention can include one of the biometric data-measuring instruments described above including a radio communication unit, and a printer that performs a radio communication with the radio communication unit to print the measured biometric data.

According to this configuration, since the printer performs a radio communication with the radio communication unit to print the measured biometric data, the measured biometric data can be reliably recorded. Therefore, even when the biometric data display unit is difficult to confirm visually, the measured biometric data can be reliably ascertained.

A muscle strength meter of the present invention includes an attachment that contacts against a living organism, and a muscle strength meter main body that is attached to the attachment, detects pressure from the living organism via the attachment, and measures the muscle strength of the living organism. The attachment includes an contacting seat part with an extending contacting face that contacts against the living organism, and an attaching shaft part for attaching the attachment to the muscle strength meter main body. The muscle strength meter main body also includes an insertion hole that the attaching shaft part is inserted into, a pressure sensor provided at a tip of the insertion hole, and a muscle strength display unit that displays the measured muscle strength. That is, it is possible to adjust the relative angle between the attachment and the muscle strength meter main body when seen from the axis direction of the attaching shaft part.

According to this configuration, since the attachment is attached to the muscle strength meter main body and includes the muscle strength display unit for displaying muscle strength, there is no need to provide a separate device including the muscle strength display unit. The whole configuration of the muscle strength meter can thus be made compact. Therefore, the portability of the muscle strength meter can be enhanced.

Moreover, since it is possible to adjust the relative angle between the attachment and the muscle strength meter main body when viewed from the long direction of the muscle strength display unit, it is easier to make the contacting seat part contact the living organism. Therefore, even when the point to be measured is one that is difficult to confirm visually with the muscle strength display unit, since considerable adjustment can be made to the relative angle between the living organism and the muscle strength meter main body, reduction of visibility can be suppressed.

A hexagonal positioning part can be provided on the attaching shaft part, with the cross-sectional shape of the insertion hole also being hexagonal.

According to this configuration, since the hexagonal positioning part is provided on the attaching shaft part, and the cross-sectional shape of the insertion hole is hexagonal, the attachment can be easily positioned to a rotation position around the axis of the insertion hole. Furthermore, since the attachment can be prevented from moving when it receives pressure from the living organism, the biometric data can be measured more precisely.

A top face of the muscle strength meter main body with its back to a face where the insertion hole is formed can be dome-shaped, and a plurality of protrusions for belt for passing a belt through can be formed on the top face. The tips of the protrusions for belt and a vertex of the rear-face dome-shape are set at the same height.

According to this configuration, by passing a belt through the protrusions for belt, the muscle strength meter main body is made easier to grasp. Furthermore, since the tips of the protrusions for belt and the vertex part of the top face of the muscle strength meter main body are set at the same height, when the top-face side is placed on a flat surface, the protrusions for belt and the vertex part can stably support the pressure from the living organism.

A muscle strength measuring system according to the present invention includes: one of the muscle strength meters described above including a radio communication unit, and a printer that performs a radio communication with the radio communication unit to print a measured muscle strength of the living organism.

According to this configuration, since the printer performs a radio communication to print the measured muscle strength, the measured muscle strength can be reliably recorded. Even if the muscle strength display unit is difficult to confirm visually, the muscle strength can be reliably ascertained.

According to the present invention, the portability of the biometric data-measuring instrument can be enhanced.

### BRIEF DESCRIPTION OF THE DRAWINGS

FIG. 1 is a schematic constitutional perspective view of a biometric data-measuring system according to a first embodiment of the invention.

FIG. 2 is a block diagram of a biometric data-measuring system according to a first embodiment of the invention.

FIG. 3 is a schematic constitutional perspective view of a biometric data-measuring instrument according to a first embodiment of the invention.

FIG. 4 is a front view of a supporting cylinder part according to a first embodiment of the invention.

FIG. 5 is a side cross-sectional view of a probe according to a first embodiment of the invention, and illustrates a state where an auxiliary cylinder part is arranged in a common plane position.

FIG. 6 is an exploded perspective view of a probe according to a first embodiment of the invention.

FIG. 7 is a view of an auxiliary cylinder part according to a first embodiment of the invention, seen from the direction of the arrow in FIG. 6.

FIG. 8 is a side cross-sectional view of a probe according to a first embodiment of the invention, and illustrates a state where an auxiliary cylinder part is arranged in a rearward refracted position.

FIG. 9 is a perspective view of a biometric data-measuring instrument according to a first embodiment of the invention when used as a muscle hardness meter, and illustrates a state where an auxiliary cylinder part is arranged in a same-plane position.

FIG. 10 is a perspective view of a biometric data-measuring instrument according to a first embodiment of the invention when used as a tenderness meter, and illustrates a state where an auxiliary cylinder part is arranged in a rearward retreated position.

FIG. 11 is a perspective view of a biometric data-measuring instrument according to a first embodiment of the invention when used as a tenderness meter, and illustrates a state where an auxiliary cylinder part is arranged in a rearward retreated position.

FIG. 12 is a plan view of a tip cap according to a first embodiment of the invention.

FIG. 13 is a perspective view of a tip cap according to a first embodiment of the invention.

FIG. 14 is a plan view of a detachable extending part according to a first embodiment of the invention.

FIG. 15 is a perspective view of a detachable extending part according to a first embodiment of the invention.

FIG. 16 is an explanatory view of a state where a tip cap according to a first embodiment of the invention is arranged in a through hole of a detachable extending part.

FIG. 17 is an explanatory view of a state where a tip cap and a detachable extending part are rotated in relation to each other, and then locked together.

FIG. 18 is an explanatory view of a state where a tip cap and a detachable extending part according to a first embodiment of the invention are seen from a side.

FIG. 19 is an explanatory view of a state where a tip cap and a detachable extending part according to a first embodiment of the invention are seen from a side.

FIG. 20 is a first explanatory view of a reading method of a biometric data display part according to a first embodiment of the invention.

FIG. 21 is a second explanatory view of a reading method of a biometric data display part according to a first embodiment of the invention.

FIG. 22 is a plan view of a modified example of a detachable extending part according to a first embodiment of the invention.

FIG. 23 is a schematic constitutional perspective view of a muscle strength-measuring system according to a second embodiment of the invention.

FIG. 24 is a block diagram of a muscle strength-measuring system according to a second embodiment of the invention.

FIG. 25 is a front view of a muscle strength meter according to a second embodiment of the invention.

FIG. 26 is a side view of a muscle strength meter according to a second embodiment of the invention.

FIG. 27 is a top view of a muscle strength meter according to a second embodiment of the invention.

FIG. 28 is a side view of an attachment according to a second embodiment of the invention.

FIG. 29 is a schematic constitutional cross-sectional view of a muscle strength meter main body according to a second embodiment of the invention.

FIG. 30 is a perspective view of a supporting part 131 according to a second embodiment of the invention.

FIG. 31 is an explanatory view of a state where an attaching shaft part 126 according to a second embodiment of the invention is inserted into an insertion hole 111, and a spherical part 136 is arranged in a protruding position and fitted into a recess 130.

FIG. 32 is a cross-sectional view of an attachment and a muscle strength meter main body according to a second embodiment of the invention.

### BEST MODE FOR CARRYING OUT THE INVENTION

Embodiments of the invention will be explained with reference to the drawings. The embodiments are intended to be illustrative in order to further understanding of the main points of the invention, and unless stated otherwise are not intended to restrict the invention only to these embodiments.

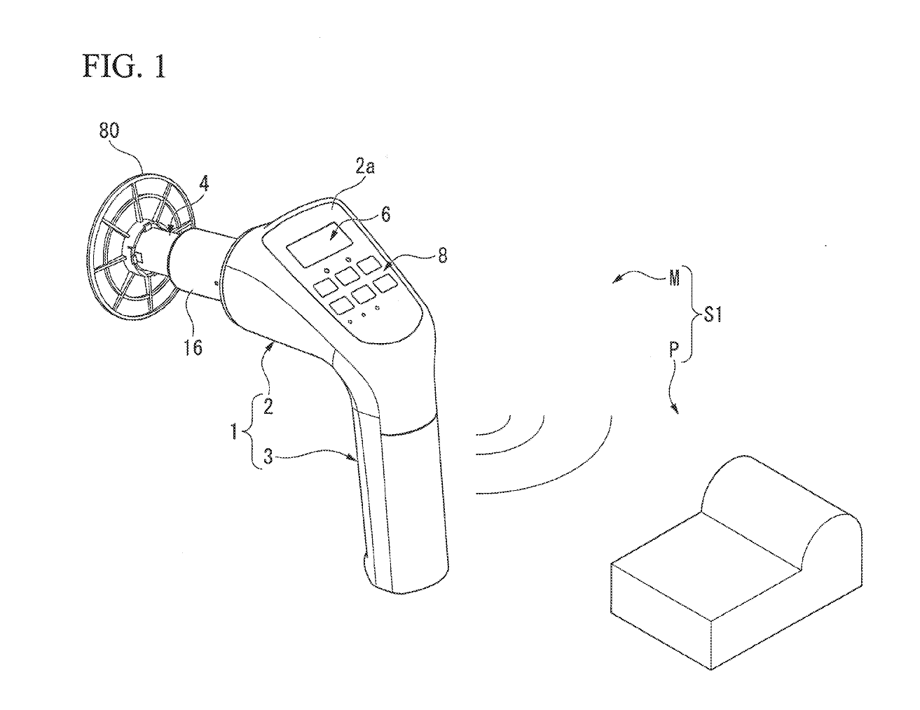

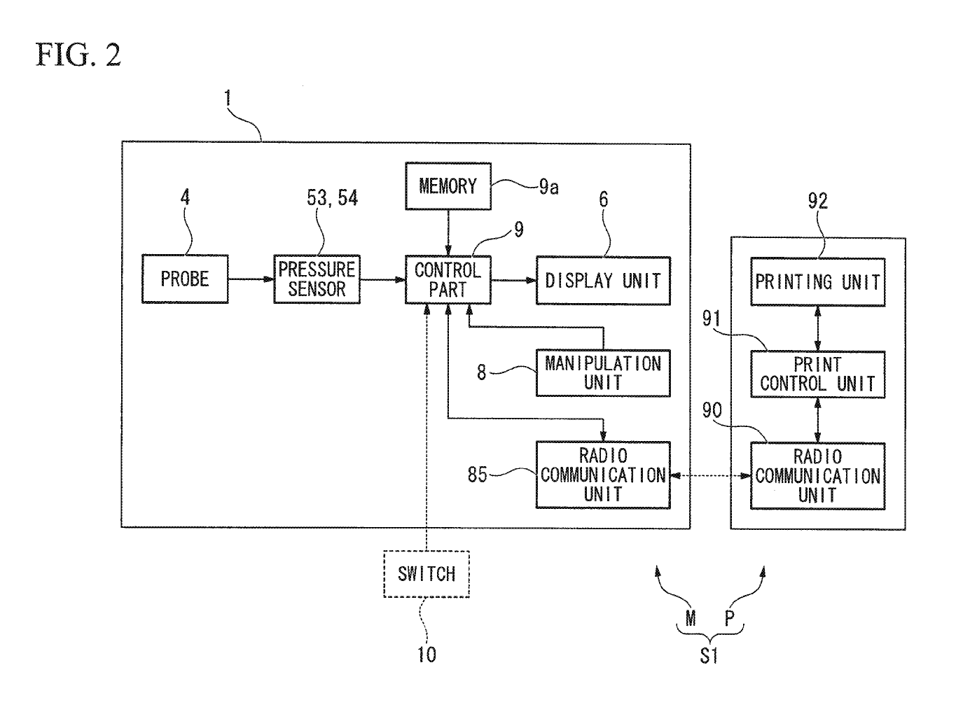

FIG. 1 is a schematic constitutional perspective view of a biometric data-measuring system 51 according to a first embodiment of the invention. FIG. 2 is a block diagram of the biometric data-measuring system 51. As shown in FIGS. 1 and 2, the biometric data-measuring system 51 includes a biometric data-measuring instrument M and a printer P.

As shown in FIG. 2, the biometric data-measuring instrument M includes a casing 1, a probe 4, a biometric data display unit 6, a manipulation part 8, a control part 9, a first pressure sensor 54, a second pressure sensor 53, and a radio communication unit 85.

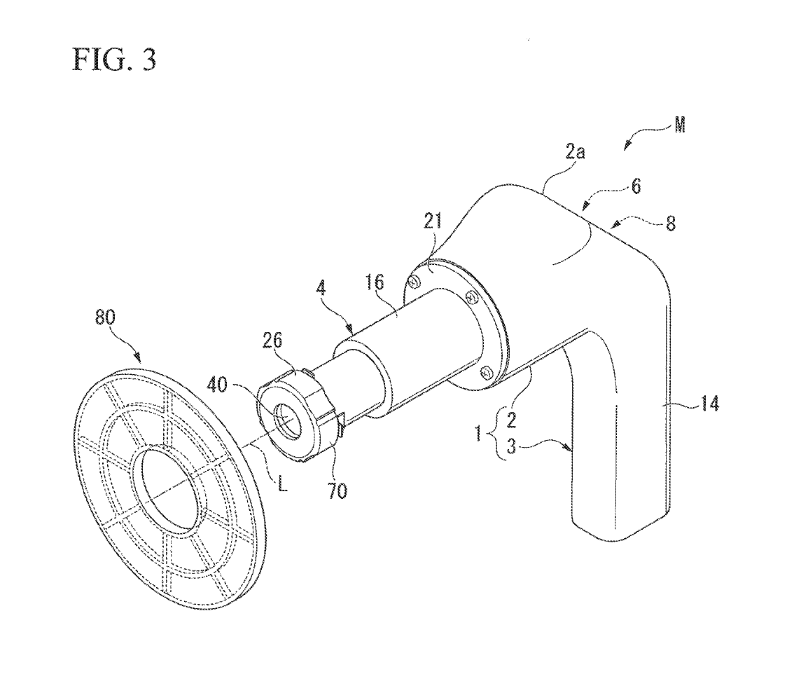

FIG. 3 is a schematic constitutional perspective view of the biometric data-measuring instrument M. As shown in FIGS. 1 and 3, the casing 1 holds the probe 4, and accommodates electronic components constituting a memory 9a and the control part 9, the biometric data display unit 6, and the manipulation part 8. The casing 1 is made from synthetic resin and formed substantially in an L-shape, and its cross-sectional contour is elliptical. The casing 1 includes a casing main-body part 2 that holds the probe 4, and a grasp part 3 that curves from the base of the casing main-body part 2 and extends in a direction intersecting the casing main-body part 2.

The casing main-body part 2 is cylindrical, and is open-ended at its tip. As shown in FIG. 1, in the outer surface of the casing main-body part 2 on the reverse side of the grasp part 3, a flat display manipulation face 2a extends from the vicinity of the tip to the base. The display manipulation face 2a inclines upwards from the base side towards the tip side. The axis L of the casing main-body part 2 becomes the pressure direction against the living organism. The pressure direction incorporates the direction of applying pressure against the living organism and the direction of receiving pressure from the living organism.

As shown in FIGS. 1 and 3, the grasp part 3 is an elongated portion which extends from the base of the casing main-body part 2 in a direction intersecting the axis L. The grasp part 3 is formed in a single body with the casing main-body part 2.

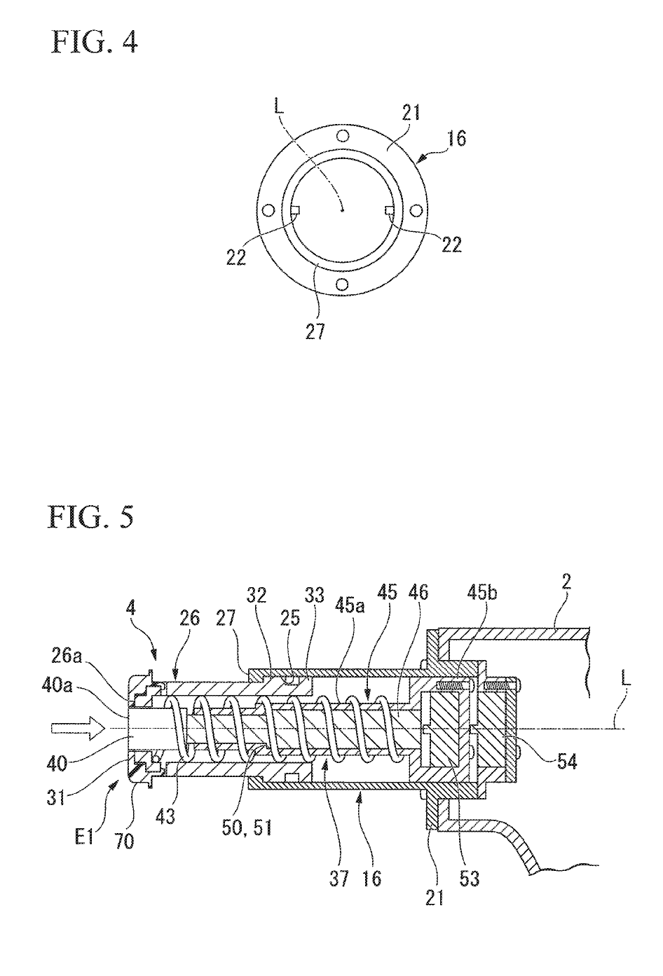

FIG. 4 is a front view of a supporting cylinder part 16, and FIG. 5 is a side cross-sectional view of the probe 4. As shown in FIG. 5, the probe 4 includes a supporting cylinder part 16, an auxiliary cylinder part 26, and a main needle part 37.

As shown in FIG. 3, the supporting cylinder part 16 is a cylindrical portion extending along the axis L, and is provided at the open end (tip) of the casing main-body part 2. A base flange 21 is provided at a base of the supporting cylinder part 16, and is extended to the radially outer side of the supporting cylinder part 16. The base flange 21 is secured in a state of covering the open end of the casing main-body part 2, and the supporting cylinder part 16 is then attached.

As shown in FIG. 4, protrusions (locking mechanisms) 22 are provided on an inner peripheral face of the supporting cylinder part 16, and protrude radially inward. The protrusions 22 are arranged opposite each other with the central axis of the supporting cylinder part 16 (axis L) between them. As shown in FIG. 5, a tip flange 27 is provided at a tip of the supporting cylinder part 16, and faces radially inward.

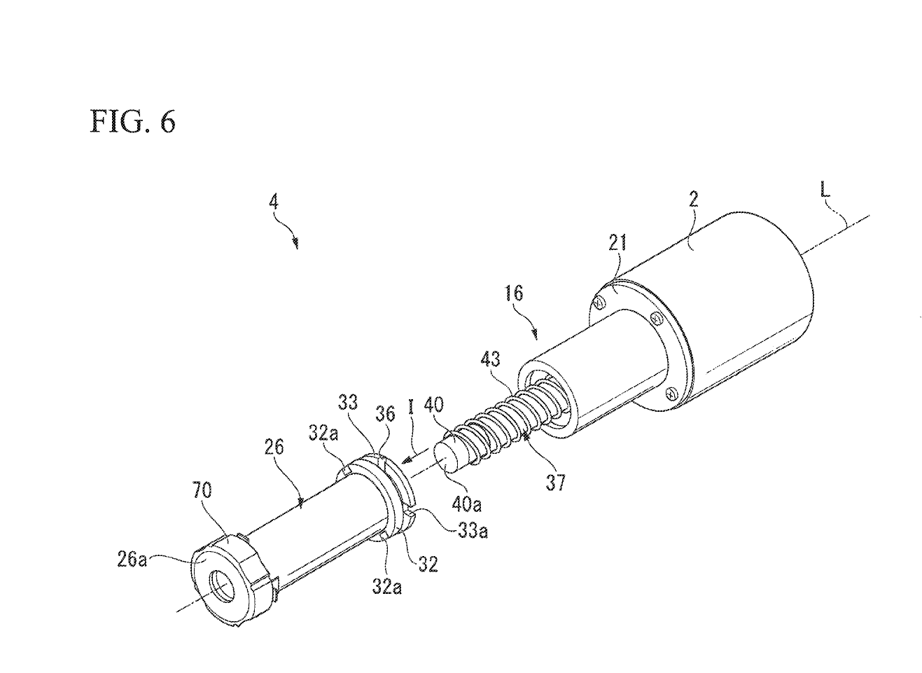

FIG. 6 is an exploded perspective view of the probe 4. As shown in FIGS. 5 and 6, the auxiliary cylinder part 26 is a cylindrical portion extending along the axis L, and is inserted into a cylindrical hole 25 in the supporting cylinder part 16. The supporting cylinder part 16 is supported such that it can reciprocate in the axis L direction.

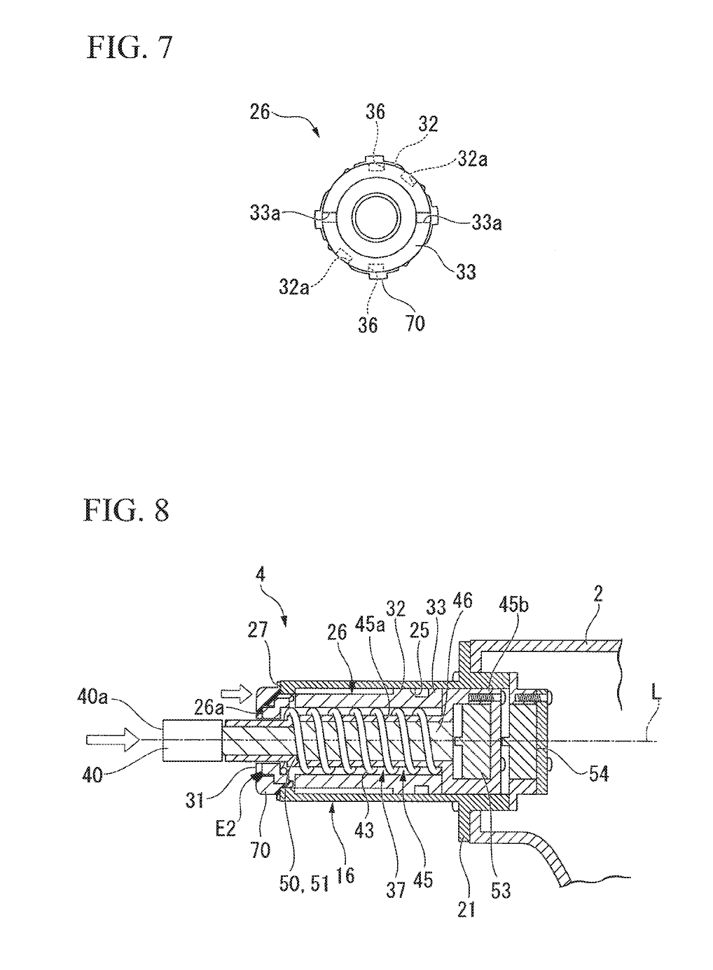

FIG. 7 is a view along arrow I in FIG. 6. As shown in FIGS. 6 and 7, a first flange part 33 is provided at the base of the outer peripheral face of the auxiliary cylinder part 26, and protrudes radially outward. As shown in FIG. 6, a first recess 33a is formed in the first flange part 33, and sinks radially inward in a rectangular shape. As shown in FIG. 7, two first recesses 33a are provided at equal intervals in the circumferential direction of the auxiliary cylinder part 26. That is, the first recesses 33a oppose each other with the central axis of the auxiliary cylinder part 26 between them. As shown in FIG. 6, the first recesses 33a are formed along the whole length of the first flange part 33 in the axis L direction.

As shown in FIG. 6, a recess for engagement (locking mechanism) 36 is formed in the first flange part 33, and sinks rearward in the axis L direction. As shown in FIG. 7, two recesses for engagement 36 are provided at equal intervals in the circumferential direction of the auxiliary cylinder part 26. That is, the recesses for engagement 36 oppose each other with the central axis of the auxiliary cylinder part 26 between them. That is, as shown in FIG. 7, the first recesses 33a and the recesses for engagement 36 are provided alternately at equal intervals in the circumferential direction.

As shown in FIG. 6, a second flange part 32 is formed at a predetermined interval from the first flange part 33 on the tip side (axis L direction) of the supporting cylinder part 16. A second recess 32a is formed in the second flange part 32, and sinks in a rectangular shape radially inward. As shown in FIG. 7, two second recesses 32a are provided at equal intervals in the circumferential direction of the auxiliary cylinder part 26. That is, the second recesses 32a oppose each other with the center point of the auxiliary cylinder part 26 between them. The second recesses 32a are formed along the whole length of the second flange part 32 in the axis L direction. The second recesses 32a are arranged at the center of the circumferential direction between the first recesses 33a and the recesses for engagement 36.

With this configuration, as shown in FIGS. 4, 6, and 7, if the auxiliary cylinder part 26 is moved in the axis L direction to a predetermined rotation position with the axis L as its center of rotation, the protrusions 22 formed inside the supporting cylinder part 16 pass the second recesses 32a. If the auxiliary cylinder part 26 is moved in the axis L direction to another rotation position, the protrusions 22 pass the first recesses 33a. Moreover, when the protrusions 22 are arranged between the first flange part 33 and the second flange part 32, if the auxiliary cylinder part 26 is rotated until the protrusions 22 match the recesses for engagement 36, the protrusions 22 engage with the recesses for engagement 36.

The first flange part 33 and the second flange part 32 have the same diameter, and both are larger than the inner diameter of the tip flange 27. That is, when the second flange part 32 is contacting against the tip flange 27, the auxiliary cylinder part 26 can be prevented from being dislocated from the supporting cylinder part 16. As shown in FIG. 5, a tip flange 31 is provided at the tip of the auxiliary cylinder part 26.

As shown in FIG. 5, the main needle part 37 is provided inside the supporting cylinder part 16 and the auxiliary cylinder part 26, and on the same axis as them. The length of the main needle part 37 is greater than the lengths of the supporting cylinder part 16 and the auxiliary cylinder part 26. Therefore, the tip of the main needle part 37 protrudes from the tip flange 27 of the supporting cylinder part 16. The main needle part 37 is supported such that it can move inside the supporting cylinder part 16 relative to the axis L direction. The main needle part 37 includes a bottomed cylindrical outer shell 45 and a cylindrical core 46. The core 46 is inserted into the outer shell 45 and supported such that it can reciprocate in the axis direction.

As shown in FIG. 5, the outer shell 45 includes a small-diameter part 45a, and a large-diameter part 45b provided at a base of the small-diameter part 45a. The small-diameter part 45a and the large-diameter part 45b are formed in a single piece. A step 50 is formed on an inner peripheral face of the small-diameter part 45a. A step 51 is formed on an outer peripheral face of the core 46. These steps 50 and 51 contact against each other, preventing the core 46 from being dislocated from the tip of the outer shell 45.

Moreover, as shown in FIG. 5, a male screw part (not shown) is formed at the tip of the core 46, and sinks to the rear end side thereof. As shown in FIGS. 5 and 6, a cylindrical tip chip (main contacting part) 40 is screwed to the tip of the main needle part 37. The tip chip 40 is thereby detachably attached to the tip of the main needle part 37.

As shown in FIG. 5, a second pressure sensor (pressure sensor) 53 made of, for example, a semiconductor, is provided in the large-diameter part 45b. When the tip chip 40 is pressed, the core 46 moves to the rear side with respect to the outer shell 45, and the second pressure sensor 53 measures the pressure of the core 46 at that time. To the rear of the second pressure sensor 53, a first pressure sensor (pressure sensor 54) is provided on the outer bottom face of the large-diameter part 45b. When the auxiliary cylinder part 26 and the tip chip 40 are pressed, the main needle part 37 moves rearward with respect to the 16, and the first pressure sensor 54 measures the pressure of the main needle part 37 at this time.

As shown in FIGS. 5 and 6, a coil spring 43 is provided around the outer peripheral of the main needle part 37. That is, the main needle part 37 is inserted inside the coil spring 43. The length of the coil spring 43 (its length when it is not elastically deforming) is larger than that of the small-diameter part 45a. The inner diameter of the coil spring 43 is larger than the outer diameter of the small-diameter part 45a and the inner diameter of the tip flange 31. That is, since the coil spring 43 is arranged between the tip face of the large-diameter part 45b and the inner face of the tip flange 31, the auxiliary cylinder part 26 is normally being urged toward the tip side, while the main needle part 37 is normally being urged toward the base side. The second flange part 32 contacts against the tip flange 27, whereby the auxiliary cylinder part 26 is kept is a state of protruding from the tip of the supporting cylinder part 16.

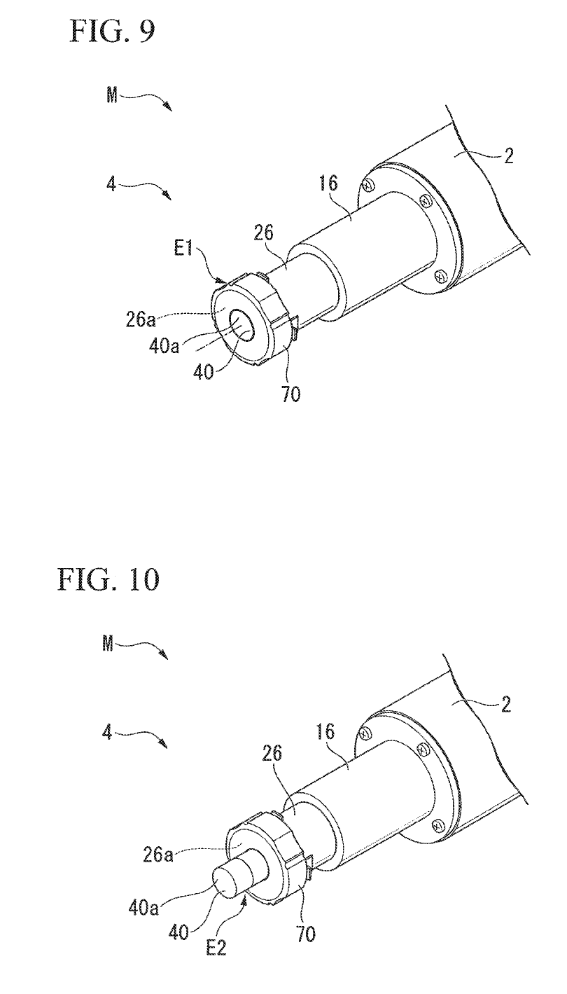

In a natural state where no external force is applied, as shown in FIGS. 5 and 9, the tip face 26a of the auxiliary cylinder part 26 (more accurately, the surface of a tip cap 70 described below) is positioned in a common plane with a tip face 40a of the tip chip 40. Let normal position E1 denote the position of the auxiliary cylinder part 26 at this time. Referring to FIG. 8, if the auxiliary cylinder part 26 is pressed toward the base side, the auxiliary cylinder part 26 resists the urging force of the coil spring 43 and moves in the direction of sinking into the supporting cylinder part 16. At this time, the auxiliary cylinder part 26 is arranged at a predetermined rotation position, and the protrusions 22 pass the second recesses 32a so that they are arranged between the first flange part 33 and the second flange part 32.

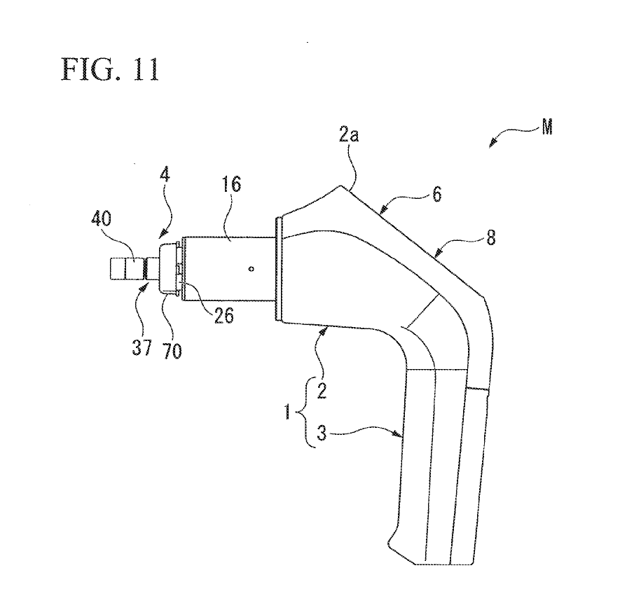

From this rotation position, the auxiliary cylinder part 26 is rotated around the axis L such that the protrusions 22 and the recesses for engagement 36 are opposite each other in the axis L direction (see FIG. 7). From this rotation position, the auxiliary cylinder part 26 is released (from the hand), the auxiliary cylinder part 26 is moved forward, and the protrusions 22 are arranged in the recesses for engagement 36. The auxiliary cylinder part 26 is thereby held in a state of being sunk in the supporting cylinder part 16. As shown in FIGS. 10 and 11, the tip face 26a of the auxiliary cylinder part 26 is at this time retracted to the rear side with respect to the tip face 40a of the tip chip 40. Let rearward position E2 denote the position of the auxiliary cylinder part 26 at this time. The auxiliary cylinder part 26 includes a tip cap 70, which is attached to its tip.

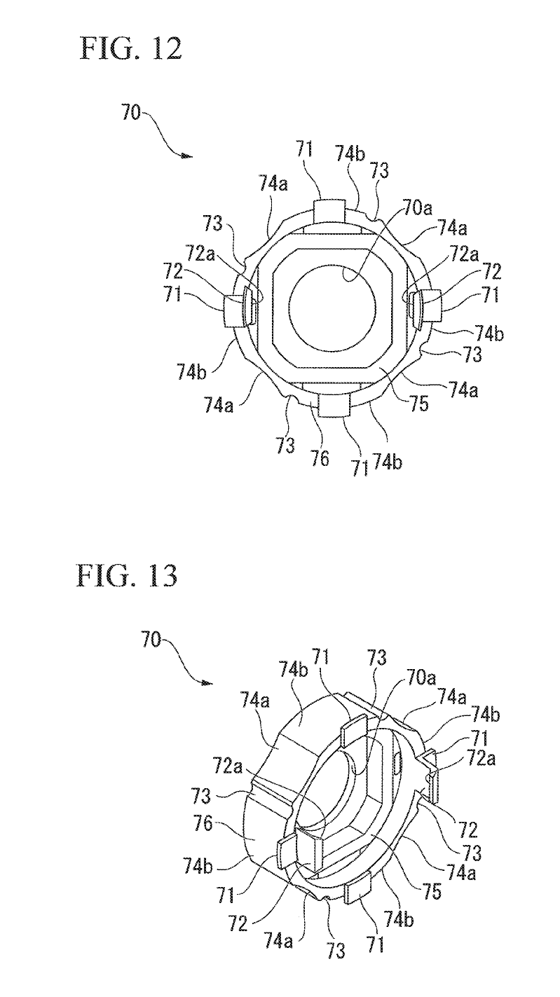

FIG. 12 is a plan view of the tip cap 70, and FIG. 13 is a perspective view of the same. As shown in FIGS. 12 and 13, the tip cap 70 includes a disk-like cap main-body part 75 formed in a ring shape, and a peripheral wall 76 rising from the whole periphery of the outer edge of the cap main-body part 75. A circular through hole 70a is formed in the center of the cap main-body part 75. The tip chip 40 is arranged in the through hole 70a (see FIGS. 5 and 9).

As shown in FIGS. 12 and 13, a peripheral wall recess 74a that sinks radially inward, and a peripheral wall projection 74b that protrudes radially outward, are formed on the peripheral wall 76. As shown in FIG. 12, four peripheral wall recesses 74a and four peripheral wall projections 74b are formed alternately at equal intervals in the peripheral direction. The peripheral wall projections 74b are formed in an arc around the center section of the cap main-body part 75, while the peripheral wall recesses 74a gently bend radially inward.

A rectangular securing protrusion (securing mechanism) 71 that protrudes radially outward is provided to an end of each peripheral wall projection 74b on the supporting cylinder part 16 side in the height direction (axis L direction). As shown in FIG. 12, the securing protrusions 71 are provided in the centers of the peripheral directions of the peripheral wall projections 74b. Also, connecting walls 72 extending to the supporting cylinder part 16 side are formed on the inner peripheries of each pair of opposing peripheral wall projections 74b. Attaching parts 72a are formed at tips of the inner faces of the connecting walls 72, and protrude radially inward. The attaching parts 72a engage with recesses (not shown) in the auxiliary cylinder part 26, whereby the tip cap 70 can be attached (see FIG. 5).

A long groove (securing mechanism) 73 is formed in the outer peripheral part of each peripheral wall projection 74b, and sinks radially inward. The long groove 73 is formed along the height-directional full length of the peripheral wall projection 74b. The long groove 73 is eccentric to one end of the outer peripheral part of the peripheral wall projection 74b in the whole circumferential direction. A detachable extending part (auxiliary contacting part) 80 is detachably provided via the tip cap 70 at the tip of the auxiliary cylinder part 26.

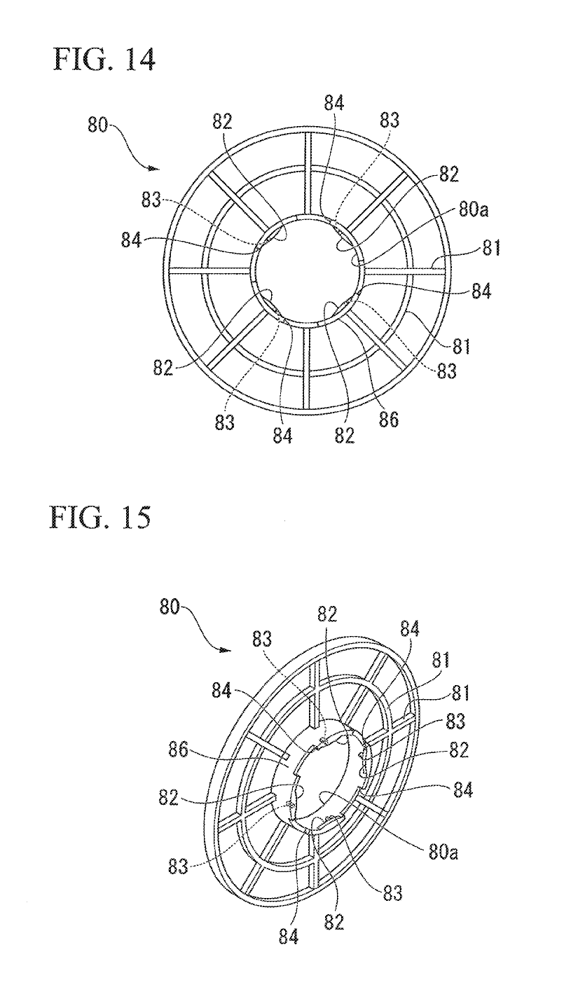

FIG. 14 is a plan view of the detachable extending part 80, and FIG. 15 is a perspective view of the same. The detachable extending part 80 is made of transparent resin, and, as shown in FIG. 3, its diameter is larger than that of the auxiliary cylinder part 26. As shown in FIG. 14, reinforcing ribs 81 are provided on the rear face of the detachable extending part 80. A through hole 80a is formed at the center of the detachable extending part 80 in the thick direction thereof. The auxiliary cylinder part 26 is designed such that its tip can be arranged in the through hole 80a. When the detachable extending part 80 is attached to the auxiliary cylinder part 26, the tip face 26a of the auxiliary cylinder part 26 and the surface of the detachable extending part 80 are arranged in a common plane.

As shown in FIGS. 14 and 15, a peripheral wall 86 is stood around the whole periphery of the edge of the through hole 80a. A securing protrusion (securing mechanism) 82 protrudes radially inward and is provided at the end of the height direction of the inner peripheral part of the peripheral wall 86 (the end of the standing direction of the peripheral wall 86). The securing protrusion 82 protrudes gently in an arc from the inner peripheral part of the peripheral wall 86. Moreover, as shown in FIG. 14, four of the securing protrusions 82 are provided at equal intervals in the circumferential direction.

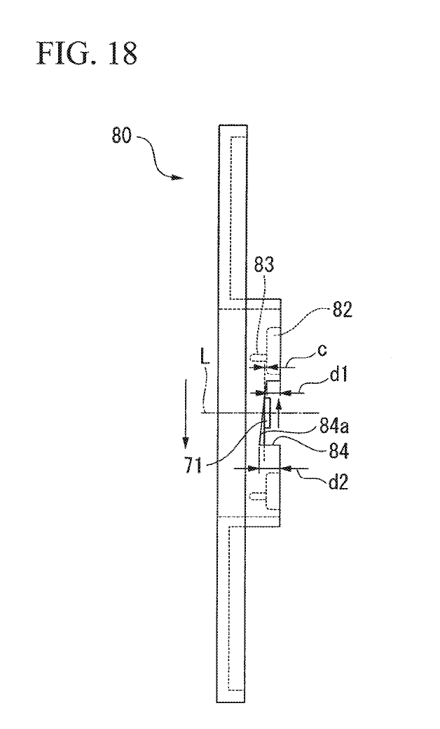

As shown in FIGS. 14 and 15, a protrusion (securing mechanism) 83 is formed on an inner peripheral part of the peripheral wall 86, and extends in the height direction of the peripheral wall 86. The protrusion 83 is eccentric to another end of the inner peripheral part of the peripheral wall 86 in the whole circumferential direction. Moreover, a notch 84 is formed in the peripheral wall 86, and sinks in a rectangular shape from the end of the height direction thereof. Four notches 84 are provided at equal intervals in the circumferential direction of the peripheral wall 86. As shown in FIG. 18, a bottom 84a of each notch 84 inclines such that its depth gradually decreases from one end of the circumferential direction to the other end (toward the securing protrusion 82 and the protrusion 83). That is, the depth d1 of one end of the notch 84 in the circumferential direction is less than the depth d2 of the other end.

Returning to FIG. 1, the biometric data display unit 6 is made of, for example, a rectangular liquid crystal, and displays various types of data and measurement values (tissue hardness and tenderness) of biometric data inputted from the control part 9. The biometric data display unit 6 is provided on the display manipulation face 2a on the tip side of the casing main-body part 2. As described above, the display manipulation face 2a inclines such as to rise from the base side toward the tip side. Therefore, as shown in FIGS. 1 and 11, the display manipulation face 2a is facing in the pressure-receiving direction in which the auxiliary cylinder part 26 receives pressure from the living organism, and in an opposite direction to the direction in which the grasp part 3 extends.

The manipulation part 8 includes manipulation buttons and the like for performing various types of manipulations, and, as shown in FIG. 2, is designed such that a person who is measuring can input his desired movement information to the control part 9. As shown in FIG. 1, the manipulation part 8 is provided on the display manipulation face 2a on the base side of the casing main-body part 2.

The control part 9 performs the following processes in accordance with muscle hardness measurement mode and muscle measurement mode. When muscle hardness measurement mode is set, the control part 9 reads measurement signals outputted from the second pressure sensor 53 and the first pressure sensor 54, and successively displays their respective measurement value information in the biometric data display unit 6. The control part 9 then reads threshold information stored in a memory 9a, and compares the threshold information with the measurement value information of the first pressure sensor 54. If the control part 9 judges that the measurement value information of the first pressure sensor 54 has exceeded the threshold information, it stores the measurement value information of the second pressure sensor 53 at that time in the memory 9a. The memory 9a also reads the measurement signal outputted in accordance with the pressing force against the second pressure sensor 53, and successively displays its measurement value information in the biometric data display unit 6. The control part 9 then reads a response signal outputted from a switch 10, and stores measurement value information at the time of reading the response signal in the memory 9a. In each of the measurement modes, the control part 9 makes the radio communication unit 85 output a radio signal indicating the measurement value information stored in the memory 9a.

The switch 10 can switch on and off, and is connected to the control part 9 via a cable (not shown). When in a released natural state, the switch 10 is off and does not output a response signal. When the person who is measuring presses the switch 10, it switches on and outputs a response signal.

The radio communication unit 85 complies with, for example, the Bluetooth specification. Based on a signal inputted from the control part 9, the radio communication unit 85 outputs a radio signal indicating the same measurement value information as that stored in the memory 9a.

As shown in FIG. 2, the printer P includes a radio communication unit 90, a print control part 91, and a printing unit 92. The radio communication unit 90 receives a radio signal outputted from the radio communication unit 85, and outputs an output signal based on this received signal to the print control part 91. Based on a signal inputted from the radio communication unit 90, the print control part 91 outputs print data to the printing unit 92. Based on the print data inputted from the print control part 91, the printing unit 92 prints measurement value information on a printing medium such as heat-sensitive paper.

Subsequently, a method of using the biometric data-measuring instrument M and the biometric data-measuring system 51 described above will be explained.

Firstly, when using the biometric data-measuring instrument M as a muscle hardness meter, as shown in FIGS. 5 and 9, the auxiliary cylinder part 26 is arranged at the normal position E1. The tip face 40a of the tip chip 40 and the tip face 26a of the auxiliary cylinder part 26 are made to contact against a point to be measured, and the biometric data-measuring instrument M is pressed against it. As a result, as shown in FIGS. 8 and 10, while the tip face 26a of the auxiliary cylinder part 26 is applying tension to the skin, the tip chip 40 is pressed into the skin. According to the reaction at this time, a rearward pressing force acts on the tip chip 40 and the auxiliary cylinder part 26.

The pressing force against the tip chip 40 is applied directly to the main needle part 37. That is, the pressing force against the tip chip 40 is applied to the core 46. The core 46 consequently moved rearward with respect to the outer shell 45, and a pressing force is applied to the second pressure sensor 53. At this time, the second pressure sensor 53 outputs a measurement signal in accordance with the pressing force. The pressing force against the second pressure sensor 53 is also applied to the outer shell 45.

In addition, the pressing force against the auxiliary cylinder part 26 is indirectly applied via the coil spring 43 to the main needle part 37. Consequently, the main needle part 37 moves rearward with respect to the supporting tube part 16, and a pressing force is applied to the first pressure sensor 54. The first pressure sensor 54 outputs a measurement signal in accordance with the pressing force at that time.

The control part 9 reads the measurement signals outputted from the second pressure sensor 53 and the first pressure sensor 54, and successively displays their respective measurement value information on the biometric data display unit 6. At this time, since the biometric data display unit 6 faces in an opposite direction to the pressure-receiving direction and the direction in which the grasp part 3 extends, it is easy for the person who is measuring to visually confirm the hardness of the muscle displayed on the biometric data display unit 6.

The control part 9 then reads the threshold information stored in the memory 9a, and compares it with the measurement value information of the first pressure sensor 54. When the control part 9 judges that the measurement value information of the first pressure sensor 54 has exceeded the threshold information, it stores the measurement value information of the second pressure sensor 53 at that time in the memory 9a. Thus the muscle hardness is measured and stored.

The control part 9 makes the radio communication unit 85 output a radio signal indicating the measurement value information when it judged that the measurement value information had exceeded the threshold information. The printer P that receives this radio signal prints the muscle hardness indicated by the radio signal on heat-sensitive paper.

On the other hand, when using the biometric data-measuring instrument M as a tenderness meter, as shown in FIGS. 8, 10, and 11, the auxiliary cylinder part 26 is arranged and locked in the rearward position E2. That is, as described above, the protrusions 22 engage with the recesses for engagement 36. This makes the tip chip 40 protrude from the tip face 26a of the auxiliary cylinder part 26. The person being measured grasps the switch 10. In this state, he contacts the tip chip 40 against a point to be measured and pushes the biometric data-measuring instrument M. According to the reaction, a rearward pressing force is applied to the tip chip 40.

The pressing force against the tip chip 40 is applied directly to the core 46. Consequently, the core 46 moves rearward with respect to the outer shell 45, and a pressing force is applied to the second pressure sensor 53. At this time, the second pressure sensor 53 outputs a measurement signal in accordance with this pressing force. The control part 9 reads the measurement signal, and successively displays the measurement value information on the biometric data display unit 6. At the moment when the person being measured feels pain, he presses the switch 10, making the switch 10 output a response signal. The control part 9 reads this response signal, and displays response information on the biometric data display unit 6. The response information is displayed in a textual or diagrammatic format. In addition, the control part 9 stores the measurement value information at the time of reading the response signal in the memory 9a. Thus the tenderness is measured and stored.

The control part 9 makes the radio communication unit 85 output a radio signal indicating the measurement value information at the time of reading the response signal. The printer P receives this radio signal, and prints the tenderness indicated by the radio signal on heat-sensitive paper.

To cancel the lock of the auxiliary cylinder part 26, the auxiliary cylinder part 26 is pushed to the rear side, the protrusions 22 are moved from the recesses for engagement 36, and the auxiliary cylinder part 26 is rotated around the axis L. When the protrusions 22 match the second recesses 32a, the auxiliary cylinder part 26 is released. The urging force of the coil spring 43 then pushes the auxiliary cylinder part 26 forward, and the protrusions 22 pass the second recesses 32a, holding the auxiliary cylinder part 26 in the normal position E1.

In using the biometric data-measuring instrument M as a muscle hardness meter, if the biometric data display unit 6 is placed at a point which is not easily visible to the person who is measuring, visibility can be adjusted in the following manner.

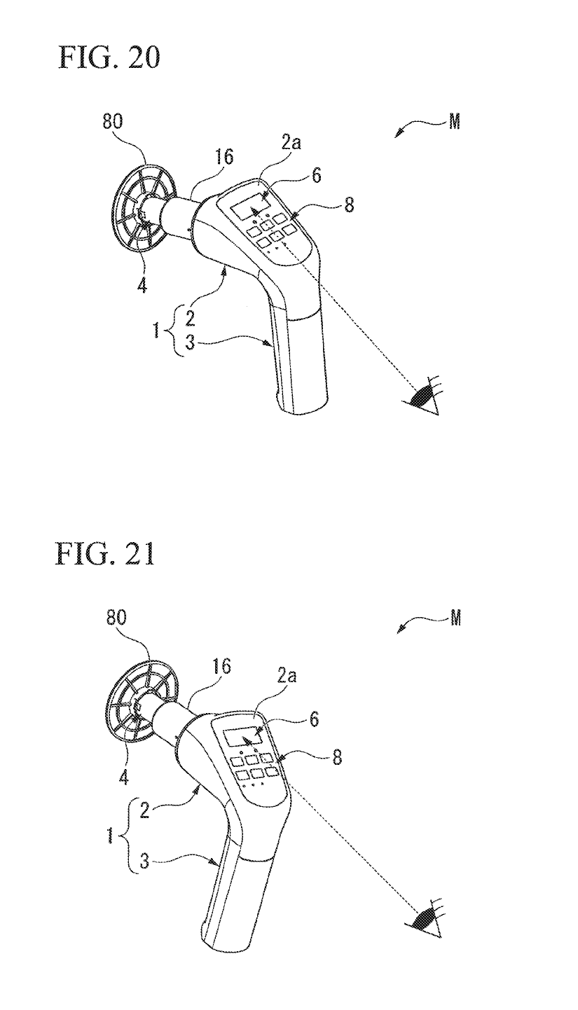

Firstly, the detachable extending part 80 is attached to the auxiliary cylinder part 26. The detachable extending part 80 is thereby arranged concentrically with the auxiliary cylinder part 26, and the surface of the detachable extending part 80 is arranged in a common plane with the tip face 26a of the auxiliary cylinder part 26. In this state, if the detachable extending part 80 is contacted against the living organism and then pushed, the increase in the contact area with the living organism makes it easier to contact against the point to be measured. That is, as shown in FIG. 20, even when the biometric data display unit 6 is facing the direction of sight of the person who is measuring, making it difficult for him to visually confirm the biometric data display unit 6, as shown in FIG. 21, the biometric data display unit 6 can be adjusted to a more easily visible position by changing the orientation of the biometric data-measuring instrument M

When the auxiliary cylinder part 26 is attached to the detachable extending part 80, the contact area with the living organism increases, thereby dispersing the load on the living organism. Consequently, even if the point to be measured is comparatively soft, the soft part of the living organism can be detected precisely, without intrusion of the auxiliary cylinder part 26. On the other hand, if the point to be measure is hard, it can be measured precisely by removing the detachable extending part 80 and contacting the auxiliary cylinder part 26 against it.

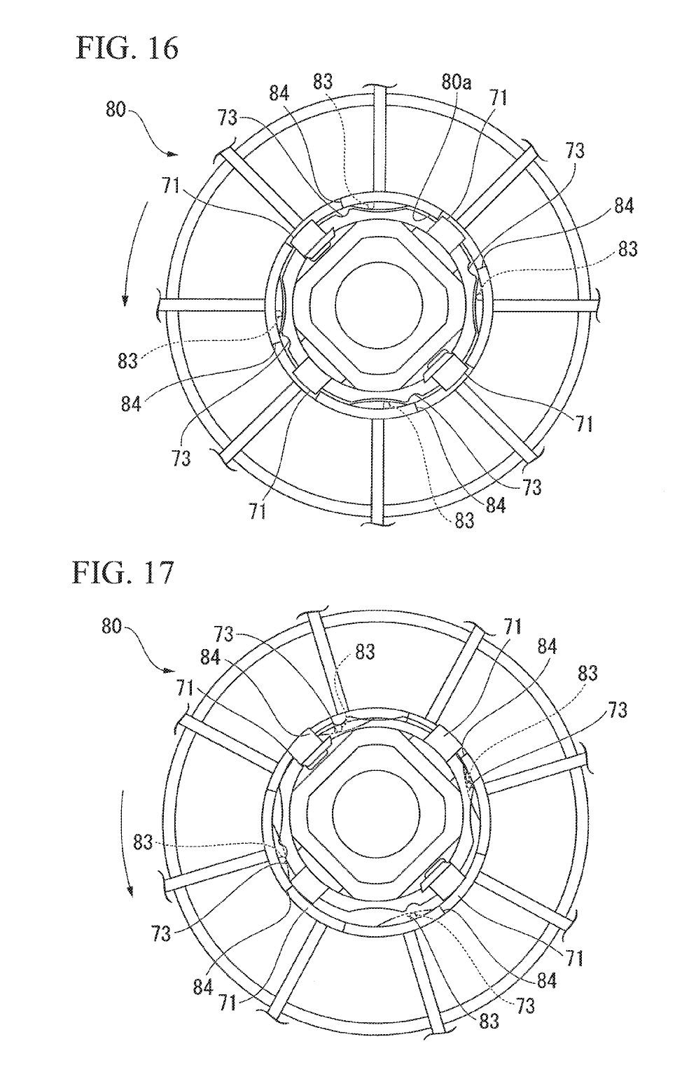

In attaching the detachable extending part 80 to the auxiliary cylinder part 26, the rotation position of the detachable extending part 80 with respect to the tip cap 70 is adjusted such that the securing protrusions 82 of the detachable extending part 80 match the peripheral wall recesses 74a of the tip cap 70. In that state, the tip cap 70 is arranged in the through hole 80a of the detachable extending part 80. As shown in FIG. 16, the securing protrusions 71 of the tip cap 70 are now contacting against the bottoms 84a of the notches 84 of the detachable extending part 80. This restricts the detachable extending part 80 from moving rearward in the axis L direction. Incidentally, when the rotation positions of the detachable extending part 80 and the tip cap 70 do not match, the securing protrusion 82 contacts against the tip face of the tip cap 70, making it impossible to attach the detachable extending part 80.

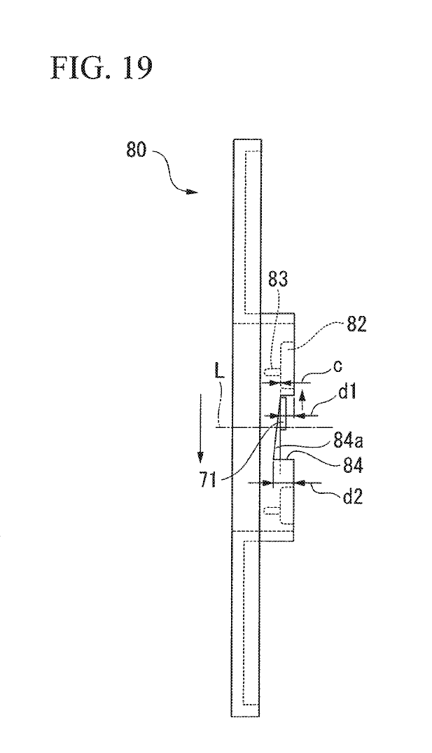

In a state where the securing protrusions 71 of the tip cap 70 are contacting against the bottoms 84a of the notches 84, as shown in FIG. 18, a clearance C is created between the axis L direction end of the tip cap 70 and the axis L direction end of the detachable extending part 80. In this state, the detachable extending part 80 is rotated in another direction (the left direction with respect to FIG. 16) and the tip cap 70 is rotated in one direction (the right direction with respect to FIG. 16). As shown in FIG. 19, the securing protrusions 71 are thereby guided by the bottoms 84a, and slide rearward in the axis L direction. That is, the tip cap 70 moves entirely rearward in the axis L with respect to the detachable extending part 80. As a consequence, a clearance C between the axis L direction end of the tip cap 70 and the axis L direction end of the detachable extending part 80 gradually decreases. Moreover, when the tip cap 70 and the detachable extending part 80 are rotated relative to each other, as shown in FIG. 17, the protrusion 83 engages with the long groove 73, restricting the relative rotation.

At this time, the axis L direction end of the tip cap 70 contacts against the axis L direction front side of the securing protrusion 82. The detachable extending part 80 is thereby restricted from moving forward in the axis L direction. As shown in FIG. 19, at this time the clearance C disappears, and, due to the contacting of the securing protrusions 71 against the bottoms 84a of the notches 84, and the contacting of the securing protrusion 82 against the axis L direction end of the tip cap 70, the detachable extending part 80 is restricted from reciprocating (locked) in the axis L direction, and it is prevented from rattling. When the detachable extending part 80 is removed, the detachable extending part 80 and the tip cap 70 need only be rotated relative to each other in the reverse direction to the one just described.

As described above, according to the biometric data-measuring instrument M, since the casing 1 includes the biometric data display unit 6 that displays measured muscle hardness and tenderness, there is no need to provide a separate device including the biometric data display unit 6. The configuration of the whole biometric data-measuring instrument M can thus be made compact. Therefore, the portability of the biometric data-measuring instrument M can be enhanced. Moreover, since the tip of the auxiliary cylinder part 26 extends further outward than the base side, the auxiliary cylinder part 26 is easily contacted against a point to be measured. Since this makes it possible to considerably adjust the relative angle of the living organism and the biometric data-measuring instrument M even if the point to be measured cannot easily be visually confirmed with biometric data display unit 6, a reduction in visibility from the biometric data display unit 6 can be suppressed.

Furthermore, since the biometric data display unit 6 is facing the pressure-receiving direction, when the person who is measuring is positioned in the pressure-receiving direction with respect to the point to be measured, he can easily visually confirm the muscle hardness and tenderness. Moreover, since the biometric data display unit 6 is facing in the opposite direction to the direction in which the grasp part 3 extends, when the biometric data display unit 6 is below the eye level of the person who is measuring, he can easily visually confirm the muscle hardness and tenderness.

Furthermore, since the casing 1 includes the grasp part 3 extending in a direction that intersects the pressure-applying direction, the orientation of the casing 1 can be stabilized by grasping the grasp part 3 such that it is in a vertical plane. This makes it possible to apply pressure stably to the point to be measured, and to more accurately measure the muscle hardness and tenderness.

Furthermore, since the detachable extending part 80 is detachably provided to the tip cap 70, the portability of the biometric data-measuring instrument M can be further enhanced. Also, the biometric data-measuring instrument M is easier to handle, enables biometric data to be measured speedily and precisely.

By rotating the detachable extending part 80 and the tip cap 70 relative to each other to make the protrusion 83 engage with the long groove 73, the securing protrusions 71 and the bottoms 84a of the notches 84 are contacted against each other; the securing protrusion 82 and the axis L direction end of the tip cap 70 are also contacted against each other. Consequently, the detachable extending part 80 and the tip cap 70 can be locked and unlocked speedily and reliably. Also, due to the notches 84, the securing protrusions 71 can be reliably contacted, and it is possible to speedily and easily lock the detachable extending part 80 and the tip cap 70. Since the bottoms 84a of the notches 84 are inclining, the securing protrusions 71 can be guided, and the detachable extending part 80 and the tip cap 70 can be reliably locked such that they do not rattle. Due to the provision of the peripheral wall recess 74a and the securing protrusion 82, the detachable extending part 80 and the tip cap 70 can easily be arranged in their appropriate rotation positions.

Due to the provision of the first flange part 33, the second flange part 32, and the protrusions 22, the auxiliary cylinder part 26 can be reliably locked with a simple configuration. Also, due to the provision of the recesses for engagement 36, the locked state can be reliably maintained.

Furthermore, since the detachable extending part 80 is mode of transparent resin, the point to be measured can be viewed through it. This enables the tip chip 40 to be contacted easily and reliably against the point to be measured.

Furthermore, since the biometric data-measuring instrument M includes a securing mechanism that secures the auxiliary cylinder part 26 on the press-applying direction side, and the switch 10, it can be made to function both as a pressure algometer and a muscle hardness meter. That is, since the auxiliary cylinder part 26 can be locked in the rearward position E2, muscle hardness and tenderness can be easily measured precisely using a single device. Further, since it can function both as a muscle hardness meter and a pressure algometer, the management load can be reduced.

Moreover, the switch 10 enables the person being measured to notify the person who is measuring the moment he feels pain. This makes it possible to precisely measure the tenderness of the person being measured. Since the measurement value information is stored according to the response signal of the switch 10, measuring can be performed precisely and easily. If the person being measured indicates the moment that he feels pain by verbal communication, a time gap arises between the moment he feels pain and the moment that he speaks, making it difficult to measure precisely. If he indicates the moment that he feels pain by movement, a time gap arises between the moment the person who is measuring sees that movement and the moment that he looks at the display on the biometric data display unit 6, making it difficult to measure precisely. That is, according to the biometric data-measuring instrument M of this embodiment, the person being measured need only press the switch 10 to easily notify the timing, whereby measuring can be performed precisely.

Furthermore, since the printer P performs a radio communication to print the measured muscle hardness and tenderness, the measured muscle hardness and tenderness can be reliably recorded. Even when the biometric data display unit 6 is difficult to confirm visually, the muscle hardness and tenderness can be reliably ascertained.

Moreover, since the tip of the auxiliary cylinder part 26 extends outwards from the base side, when measuring a hard point on the living organism, the hardness of the muscle of the living organism can be measured by contacting the auxiliary cylinder part 26 against that point. When the detachable extending part 80 is attached, the hardness of a soft point on the living organism can be measured accurately. Therefore, irrespective of the hardness of the point to be measured, the biometric data-measuring instrument of the above-described embodiment can measure the hardness of that point easily and precisely.

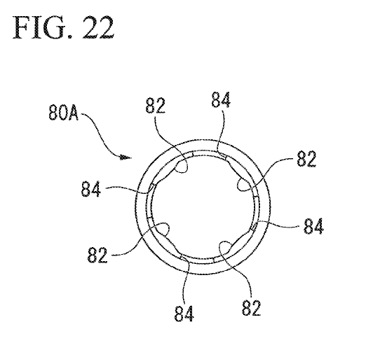

While in the above-described embodiment, one type of detachable extending part 80 is used, this is not limited. For example, a number of differently-sized types of detachable extending parts can be prepared beforehand, and tip caps of those sizes can be exchanged selectively. ‘Size’ includes not only dimensions but also shapes. As for example shown in FIG. 22, it is possible to use a detachable extending part 80A, which has a smaller diameter than the detachable extending part 80 and a larger diameter than the auxiliary cylinder part 26.

While the above-described embodiment includes the switch 10, this need not be included. It is preferable to include the switch 10, however, as this can achieve a precise measurement.

While in the above-described embodiment, the response from the switch 10 is notified using the biometric data display unit 6, this configuration is not limited and can be modified where necessary. For example, the response can be notified using sound, vibrations, etc.

While in the above-described embodiment, the printer P uses heat-sensitive paper as the printing medium for printing the measured biometric data, ordinary paper or another film-like printing medium can be used.

While in the above-described embodiment, the radio communication units 85 and 90 employ the Bluetooth specification as a radio method, they can employ another specification or an independent communication method.

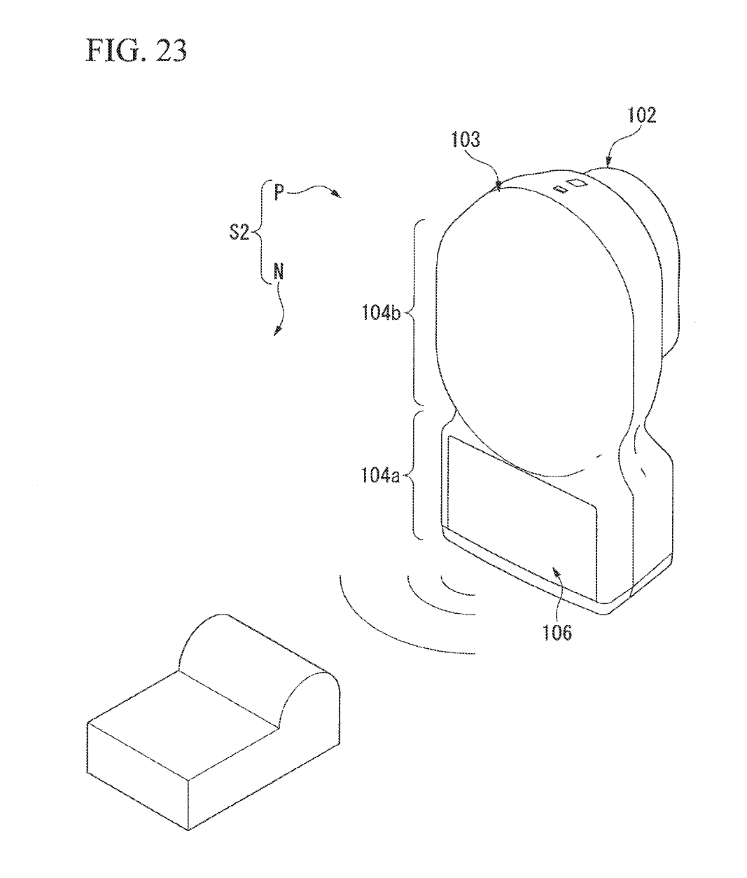

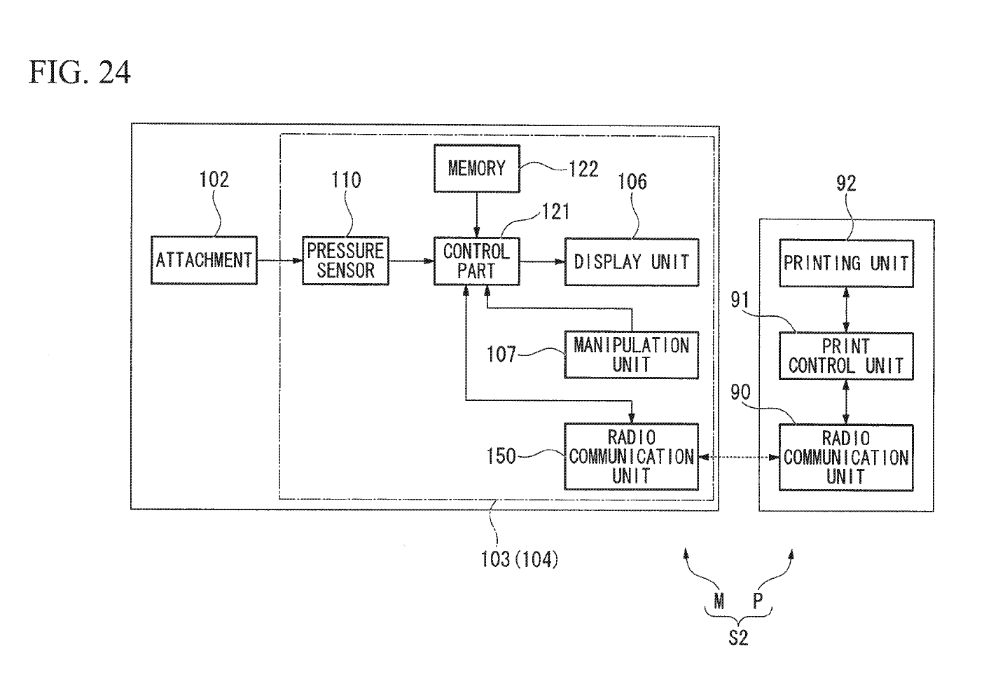

FIG. 23 is a schematic constitutional perspective view of a muscle strength-measuring system S2 according to a second embodiment. FIG. 24 is a block diagram of the muscle strength-measuring system S2. In FIGS. 23 to 32, constituent elements similar to those in FIGS. 1 to 22 are designated with like reference numerals and are not repetitiously explained.

As shown in FIGS. 23 and 24, the muscle strength-measuring system S2 includes a muscle strength meter N and a printer P.

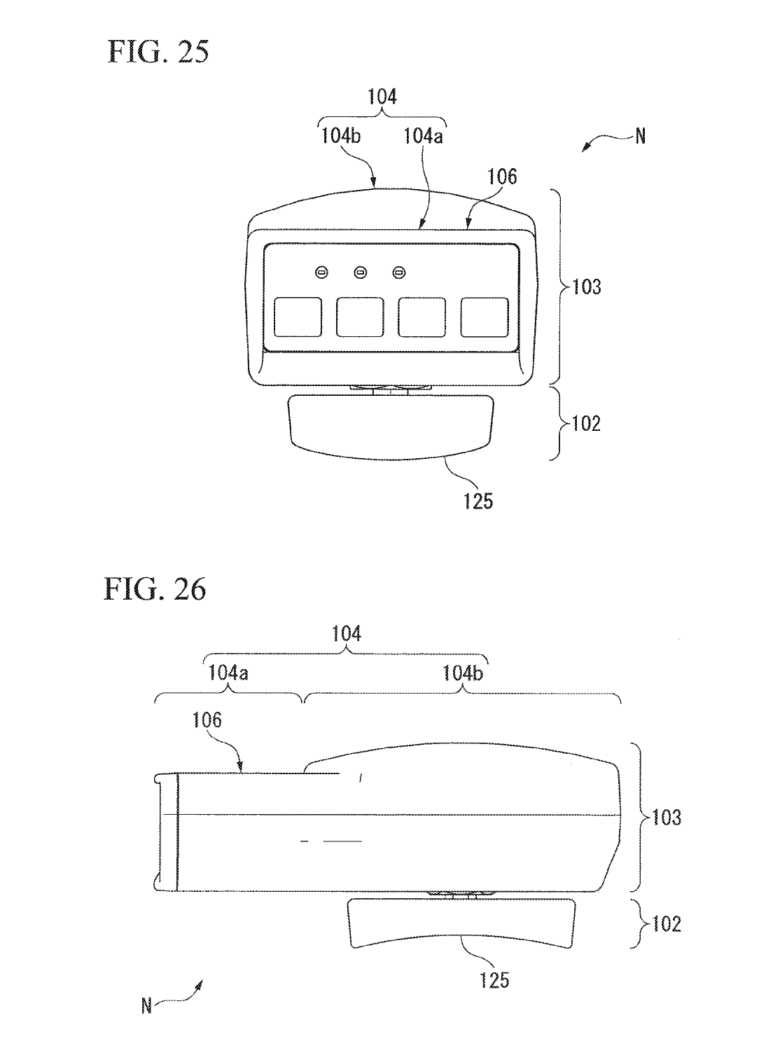

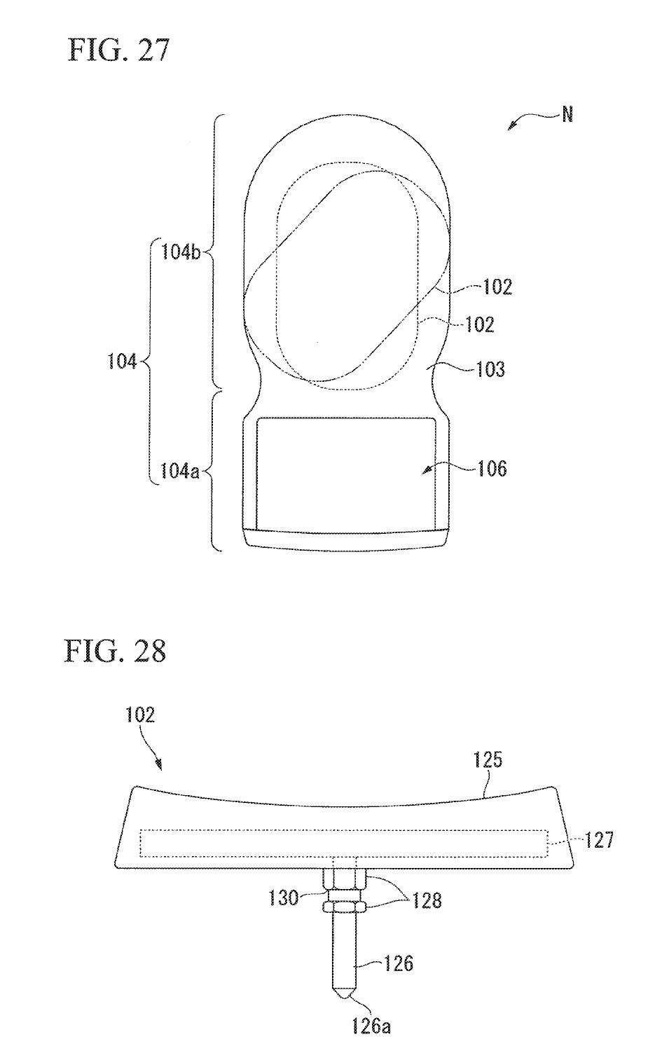

FIG. 25 is a front view of the muscle strength meter N, FIG. 26 is a side view of the same, and FIG. 27 is a top view of the same. As shown in FIGS. 25 to 27, the muscle strength meter N includes an attachment 102 that contacts against a living organism, and a muscle strength meter main body 103 that measures muscle strength based on pressure from the living organism.

FIG. 28 is a side view of the attachment 102. As shown in FIG. 28, the attachment 102 is made by insert molding, and includes an elliptical plate 127 arranged inside a contacting seat part 125 which is substantially trapezoidal in side view.

The contacting seat part 125 is made of resin, a lower bottom part and an upper bottom part of the trapezoidal shape being substantially elliptical in plan view (see FIG. 27). As shown in FIG. 28, the lower bottom part of the contacting seat part 125 curves gently to the inner side such that it becomes gradually thinner away from both ends in its long direction. Therefore, even if the contacting seat part 125 is contacted against a curving point on a protrusion of the living organism, the whole of the lower bottom face can be stably brought into intimate contact with the living organism. The upper bottom part of the contacting seat part 125 is flat.

The elliptical plate 127 is a metal plate formed in a substantially elliptical shape in plan view, and extends in the same direction as the upper bottom part. An attaching shaft part 126 is welded to a rear face of the elliptical plate 127, and extends in a direction that intersects the direction in which the elliptical plate 127 extends. A projection-side tapered part 126a having a diameter that gradually decreases as it approaches the tip is formed at a tip part of the attaching shaft part 126. That is, the tip part of the attaching shaft part 126 is tapered.

As shown in FIG. 28, hexagonal positioning parts 128 are provided at the base of the attaching shaft part 126. There are two positioning parts 128, each extending outward from the attaching shaft part 126, provided in the long direction of the attaching shaft part 126 with an interval between them. That is, a recess 130 is formed between the positioning parts 128, and sinks toward the axis of the attaching shaft part 126. This recess 130 is formed over the whole periphery of the attaching shaft part 126.

Moreover, the elliptical plate 127 is provided inside the contacting seat part 125. A connection part welded to the rear face of the elliptical plate 127 is also arranged inside the contacting seat part 125.

Returning to FIG. 24, a muscle strength meter main body 103 includes a casing 104, a pressure sensor 110, a muscle strength display unit 106, a manipulation part 107, a control part 121, a memory 122, and a radio communication unit 150.

As shown in FIGS. 23 and 24, the casing 104 has an external shape including a rectangular parallelepiped part 104a on a front-face side and a dome-like part 104b on a back-face side, these being joined in a single piece. A bottom face of the casing 104 is flat. On a top face of the casing 104, the rectangular parallelepiped part 104a is a flat display face where the muscle strength display unit 106 is provided, and the dome-shaped part 104b becomes gradually thinner as it runs from its peripheral edge toward a center point.

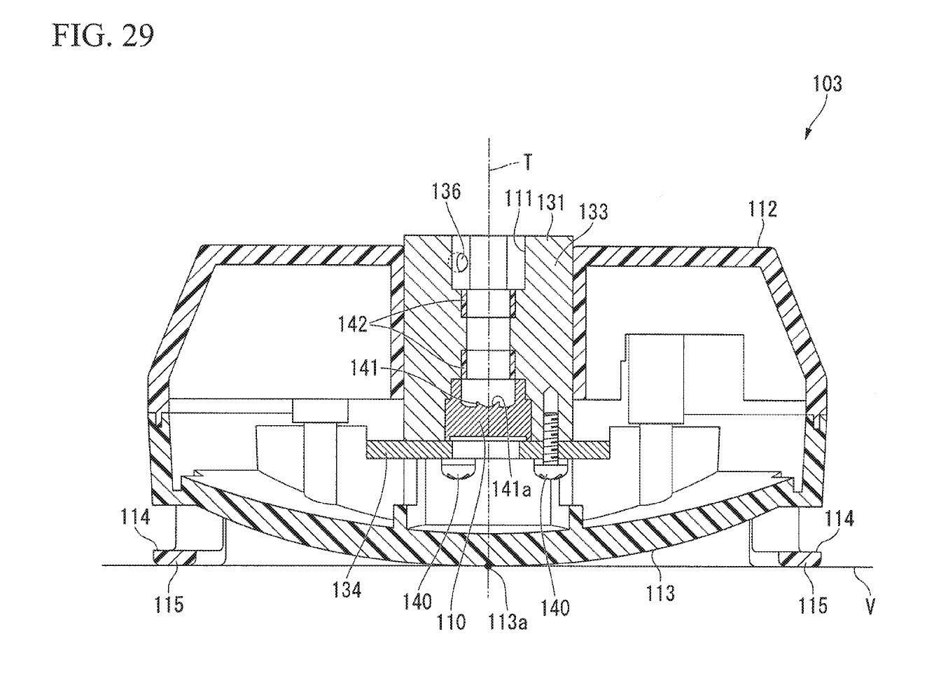

FIG. 29 is a schematic constitutional cross-sectional view of the muscle strength meter main body 103. As shown in FIG. 29, the dome-shaped part 104b includes a pair of leg parts (protrusions for belt) 115 with a slit 114 formed therein for passing a belt through so as to be easily graspable by the person who is measuring. Moreover, the tip of each leg part and the vertex part 113a of a dome 113 are set at the same height. That is, the tip of the leg part and the vertex part 113a are arranged on a straight line U that extends in a direction intersecting the axis T of an insertion hole 111 described later. A metal supporting part 131 is provided in the casing 104.

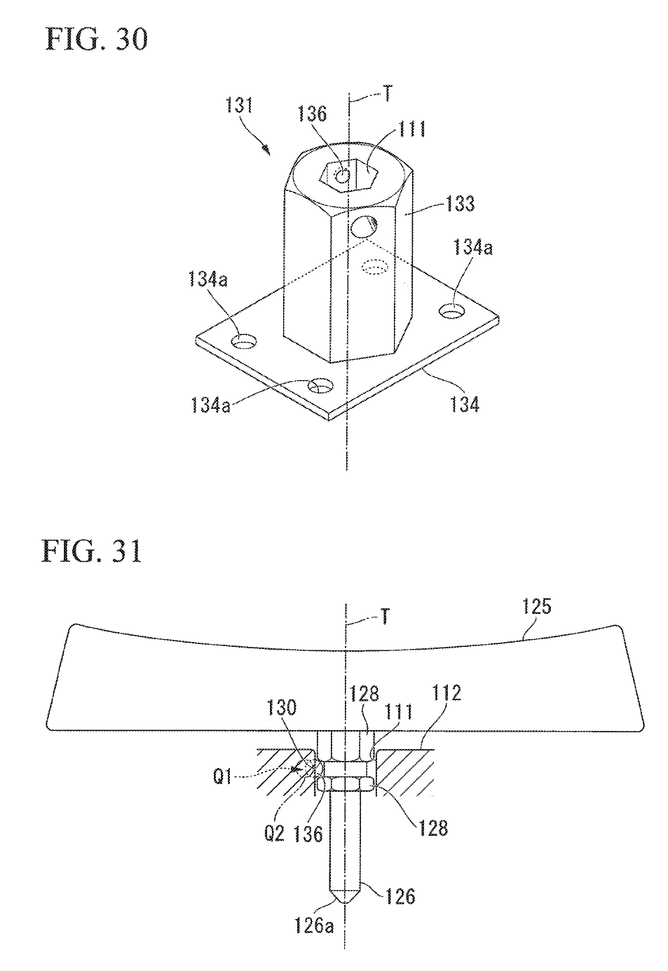

FIG. 30 is a perspective view of the supporting part 131. As shown in FIG. 30, the supporting part 131 includes a column part 133 extending in a hexagonal column shape, and a rectangular reinforcing plate 134 extending in a direction intersecting the long direction of the column part 133. The column part 133 and the reinforcing plate 134 are built into the casing 104. A tip face of the column part 133 is arranged in a common plane with the flat face 112, and exposed to the outside. As shown in FIG. 30, a plurality of attachment holes 134a are formed in the reinforcing plate 134. The supporting part 131 is secured in the casing 104 by passing securing screws 140 (shown in FIG. 29) through the attachment holes 134a.

An insertion hole 111 is formed in the column part 133, and extends in the axis T direction. The insertion hole 111 is open from the tip face of the column part 133. Also, the insertion hole 111 is concentric to the column part 133. Moreover, the cross-sectional face of the insertion hole 111 is formed in a hexagonal shape. Therefore, when the attaching shaft part 126 is inserted into the insertion hole 111, the positioning part 128 fits into the insertion hole 111, thereby positioning the attachment 102 and the muscle strength meter main body 103 in their relative rotation positions, and restricting their relative rotation. The hexagonal shape of the outer periphery of the column part 133 and the cross-sectional hexagonal shape of the insertion hole 111 are oriented in the same rotation position around the axis T of the insertion hole 111, and the external appearance of the column part 133 makes it easy to see the orientation of the insertion hole 111.

As shown in FIG. 31, a spherical part 136 that can move in a direction intersecting the axis T of the insertion hole 111 is provided at a top end of the column part 133. The spherical part 136 is supported such that it can move between a projecting position Q1, where it projects from the inner peripheral face of the insertion hole 111 toward the axis of the insertion hole 111, and a sunken position Q2, where it sinks inward from the inner peripheral face. An elastic member (not shown) is provided on the deep side of the spherical part 136, and urges the spherical part 136 toward the axis of the insertion hole 111. Incidentally, since the diameter of the opening for allowing the spherical part 136 to appear outside is smaller than the diameter of the spherical part 136, the elastic member urges the spherical part 136 and prevents it from moving outside.

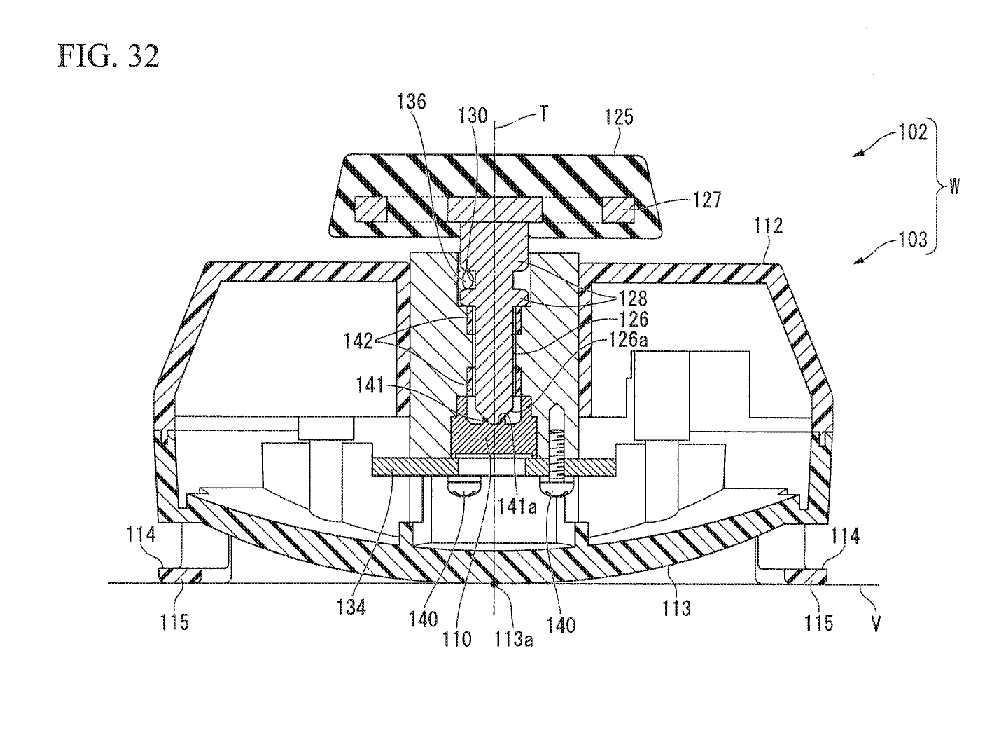

Moreover, as shown in FIG. 32, a ring part 142 is made of resin formed in a ring shape, and is provided on an inner peripheral face of the insertion hole 111. Two ring parts 142 are provided with an interval between them in the axis T direction. The inner diameters of the ring parts 142 are the same as, or smaller than, the outer diameter of the attaching shaft part 126.

The pressure sensor 110 is built into the tip of the insertion hole 111. When the attaching shaft part 126 is inserted in the insertion hole 111, the pressure sensor 110 receives the pressure from the attachment 102, detects this pressure, and outputs the detection result to the control part 121 (see FIG. 24). A guiding part 141 for guiding the attaching shaft part 126 is provided on a top face of the pressure sensor 110 (the face on the insertion hole 111 side). A recess-side tapered part 141a is formed in the guiding part 141, and, around a center point on the axis T, has a diverging degree spreading outwardly toward the open side of the insertion hole 111. That is, the recess-side tapered part 141a and the projection-side tapered part 126a of the attaching shaft part 126 have complementary recess and projection shapes, the attaching shaft part 126 being arranged concentrically in the insertion hole 111.

As shown in FIGS. 23 and 27, the muscle strength display unit 106 is made of, for example, a rectangular liquid crystal, and displays muscle strength measurement value and various types of data inputted to it from the control part 121. The muscle strength display unit 106 is provided on the flat surface of the rectangular parallelepiped part 104a on the top face of the casing 104.

As shown in FIG. 25, the manipulation part 107 includes manipulation buttons and the like for performing various types of manipulations, and is designed such that the person who is measuring can input his desired movement to the control part 121. As shown in FIG. 25, the manipulation part 8 is provided on the front face of the casing 104.

As shown in FIG. 24, the control part 121 calculates the muscle strength of the living organism from the detection result of the pressure sensor 110, and makes the muscle strength display unit 106 display the calculation result. The control part 121 also outputs a radio signal indicating calculated measurement value information to the radio communication unit 150.

The radio communication unit 150 complies with, for example, the Bluetooth specification, and, as shown in FIG. 24, based on a signal inputted from the control part 121, outputs a radio signal indicating the measurement value information.

Subsequently, a method of using the muscle strength meter N and the muscle strength-measuring system S2 in the embodiment described above will be explained.

Firstly, the attaching shaft part 126 is inserted in the insertion hole 111, and the attachment 102 is attached to the muscle strength meter main body 103 (see FIG. 29 and FIG. 32). In this state, for example, the hand or foot of the person who is measuring is contacted against the contacting seat part 125. When the person who is measuring pushes the contacting seat part 125 with his hand or his foot, the pressure is transmitted to the contacting seat part 125, and the elliptical plate 127 functions as reinforcement for receiving the pressure. The pressure transmitted to the contacting seat part 125 and the elliptical plate 127 is then transmitted through the attaching shaft part 126, passing from the tip thereof to the pressure sensor 110. The pressure sensor 110 detects the pressure at this time, and outputs a detection result to the control part 121. When the person who is measuring manipulates the manipulation part 107, the control part 121, based on the calculation result, calculates the muscle strength and displays the result on the muscle strength display unit 106.

The attachment 102 is then released from the muscle strength meter main body 103, rotated, and reattached, while adjusting the relative angle between the attachment 102 and the muscle strength meter main body 103. Thus, even when the muscle strength display unit 106 of the muscle strength meter N is at a position that is difficult to confirm visually, the muscle strength display unit 106 can be moved to a position where it can easily be seen (see FIG. 27).

In attaching the attachment 102 to the muscle strength meter main body 103, when the attaching shaft part 126 is inserted in the insertion hole 111 as far as a predetermined position, the attachment 102 is then secured to the attachment 102 in the following manner. As shown in FIG. 30, the tip of the attaching shaft part 126 is inserted from the open end of the insertion hole 111. Since the diameter of the opening in the insertion hole 111 is larger than the outer diameter of the attaching shaft part 126, the spherical part 136 is now at projecting position Q1. When the attaching shaft part 126 is pushed inward, the positioning part 128 on the tip side contacts against the spherical part 136. When the attaching shaft part 126 is pushed further, as shown in FIG. 31, the spherical part 136 moves to the inside and sinks with respect to the inner peripheral face of the insertion hole 111. That is, the spherical part 136 is at the sunken position Q2. When the attaching shaft part 126 is pushed even more, as shown in FIG. 31, the recess 130 faces the spherical part 136 in a direction intersecting the axis T. Since the spherical part 136 is urged by an urging member (not shown), it moves outward to the projecting position Q1. Consequently, the spherical part 136 fits into the recess 130, and the attachment 102 is thereby secured to the muscle strength meter main body 103.

When the attaching shaft part 126 is inserted in the insertion hole 111, due to the ring part 142 provided on the inner peripheral face of the insertion hole 111, the attaching shaft part 126 enters without contact between its metal and the metal of the inner peripheral face of the insertion hole 111, the only contact being the metal-against-resin contact between the attaching shaft part 126 and the ring part 142. Moreover, since a plurality of ring parts 142 are provided, the attaching shaft part 126 is supported at a plurality of points, and can therefore be inserted stably.

As described above, according to the muscle strength meter N, since the attachment 102 is attached to the muscle strength meter main body 103, which includes the muscle strength display unit 106 for displaying the muscle strength, there is no need to provide a separate device including the muscle strength display unit 106. The whole configuration of the muscle strength meter N can thus be made compact. Therefore, the portability of the muscle strength meter N can be enhanced. Moreover, since it is possible to adjust the relative angle between the attachment 102 and the muscle strength meter main body 103 when viewed from the long direction of the muscle strength display unit 106, it is easier to make the contacting seat part 125 contact the living organism. Therefore, even when the point to be measured is one that is difficult to confirm visually with the muscle strength display unit 106, since considerable adjustment can be made to the relative angle between the living organism and the muscle strength meter main body 103, reduction of visibility can be suppressed.

Furthermore, since the attaching shaft part 126 includes the polygonal positioning part 128, and the cross-sectional shape of the insertion hole 111 is hexagonal, the attachment 102 can easily be positioned at a rotation position around the axis of the insertion hole 111. Moreover, the attachment 102 can be prevented from moving when it receives pressure from the living organism, thereby enabling the biometric data to be measured more precisely.

By passing a belt through the leg part 115, the muscle strength meter main body 103 is made easier to grasp. Furthermore, since the tips of the leg parts 15 and the vertex part 113a are the same height, when the top-face side is placed on a flat surface, the legs 115 and the vertex part 113a can stably support the pressure from the living organism.

Furthermore, since the printer P performs a radio communication to print the measured muscle strength, the measured muscle strength can be reliably recorded. Even when the muscle strength display unit 106 is difficult to confirm visually, the muscle strength can be reliably ascertained.

Furthermore, since the column part 133 is formed in a hexagonal shape, and the orientation of insertion hole 111 is the same as that of the column part 133, when the supporting part 131 is secured in the muscle strength meter main body 103, it is easy to visually confirm the orientation of the insertion hole 111 while looking at the external appearance of the column part 133. As a result, the burden of assembling work can be reduced.

Furthermore, since the ring parts 142 are provided on the inner peripheral face of the insertion hole 111, the attaching shaft part 126 contacts the ring parts 142 when it is inserted into the insertion hole 111, making insertion easier. Moreover, since a plurality of ring parts 142 are provided, the attaching shaft part 126 can be supported at a plurality of points, whereby it can be more stably inserted into the insertion hole 111.

While in the above-described embodiment, the cross-sectional face of the insertion hole 111, the positioning part 128, and the column part 133 are hexagonal, this is not limited. These shapes can be polygonal, or another shape as appropriate.

The technological scope of the present invention is not limited to the embodiments described above, and can be modified in various ways without depart from the main points of the invention.

1. A biometric data-measuring instrument that measures data relating to a living organism by applying pressure to said living organism, comprising:
a casing;
an auxiliary contacting part that extends from said casing, the auxiliary contacting part being contacted against a vicinity of a point to be measured on said living organism and applying pressure to the vicinity of said point to be measured;
a main contacting part that, in a state where said auxiliary contacting part is applying pressure to the vicinity of said point to be measured, is contacted against said point to be measured and applies pressure to said point to be measured in the direction in which said auxiliary contacting part is applying pressure to the vicinity of said point to be measured;
a pressure sensor that is provided inside said casing and measures a pressure that said main contacting part receives from said point to be measured;
a biometric data display unit that is provided on said casing and displays said measured biometric data;
a tip of said auxiliary contacting part extending outward from a base side thereof.

2. The biometric data-measuring instrument according to claim 1, wherein said biometric data display unit is facing in the pressure-receiving direction in which said auxiliary contacting part receives pressure from said living organism.

3. The biometric data-measuring instrument according to claim 1, wherein said casing has a grasp part extending in a direction intersecting said pressure-applying direction, and said biometric data display unit is facing in an opposite direction to a direction in which the grasp part extends.

4. The biometric data-measuring instrument according to claim 1, wherein said auxiliary contacting part comprises:
a detachable extending part that is detachably provided at a tip of said auxiliary contacting part; and
a locking mechanism at a tip of this detachable extending part.

5. The biometric data-measuring instrument according to claim 1, comprising:
a securing mechanism that secures said auxiliary contacting part such that the tip of said main contacting part is positioned further to said pressure-applying direction side than the tip of said auxiliary contacting part; and
a switch that, when switched on, makes said biometric data display unit display said measured biometric data.

6. A biometric data-measuring system comprising:
the biometric data-measuring instrument according to claim 1 comprising a radio communication unit; and
a printer that performs a radio communication with said radio communication unit to print said measured biometric data.

7. A muscle strength meter comprising:
an attachment that contacts against a living organism; and
a muscle strength meter main body that is attached to said attachment, detects pressure from said living organism via said attachment, and measures the muscle strength of said living organism;
said attachment comprising:
an contacting seat part with an extending contacting face that contacts against said living organism; and
an attaching shaft part for attaching said attachment to said muscle strength meter main body;
said muscle strength meter main body comprising:
an insertion hole that said attaching shaft part is inserted into;
a pressure sensor provided at a tip of said insertion hole; and
a muscle strength display unit that displays said measured muscle strength;
it being possible to adjust the relative angle between said attachment and said muscle strength meter main body when seen from the axis direction of said attaching shaft part.

8. The muscle strength meter according to claim 7, wherein a hexagonal positioning part is provided on said attaching shaft part, and
the cross-sectional shape of said insertion hole is hexagonal.

9. The muscle strength meter according to claim 7, wherein a top face of the muscle strength meter main body with its back to a face where said insertion hole is formed is dome-shaped, and
a plurality of protrusions for belt for passing a belt through are formed on said top face;
tips of said protrusions for belt and a vertex of said rear-face dome-shape are the same height.

10. A muscle strength measuring system comprising:
the muscle strength meter according to claim 7 comprising a radio communication unit; and
a printer that performs a radio communication with said radio communication unit to print a measured muscle strength of said living organism.
